(19) United States
(12) Patent Application Publication
(10) Pub. No.: US 2014/0239658 A1
(43) Pub. Date: Aug. 28, 2014

(54) RELEASE DEVICE FOR RELEASING A MEDICAL IMPLANT FROM AN INSERTION DEVICE, AND INSERTION DEVICE COMPRISING A RELEASE DEVICE

(71) Applicant: BIOTRONIK AG, Buelach (CH)

(72) Inventors: Matthias Wesselmann, Ruedlingen (CH); Bodo Quint, Oberglatt (CH)

(73) Assignee: BIOTRONIK AG, Buelach (CH)

(21) Appl. No.: 14/177,658

(22) Filed: Feb. 11, 2014

Related U.S. Application Data
(60) Provisional application No. 61/767,800, filed on Feb. 22, 2013.

(51) Int. Cl. A61B 17/00; A61B 17/34

(52) U.S. Cl. CPC A61B 17/00234; A61B 17/3468
USPC 294/192

(57) ABSTRACT

A release device (160, 160a, 160b) for releasing a medical implant (155) from an insertion device (120), said release device comprising a body (10, 10a, 10b) with a proximal end (12) and a distal end (14), wherein a first grip segment (34, 34′), a second grip segment (34, 34′) and at least one actuator (26) are provided between the proximal and the distal end (12, 14), wherein the at least one actuator (26) is manually operable and can be brought substantially into at least the first grip segment (34) substantially axially displaceably in order to effect an intended relative movement in the longitudinal direction (22) between the first and the second insertion element (140, 145) of the insertion device (120).

### CROSS REFERENCE TO RELATED APPLICATIONS

This application claims benefit of priority to U.S. patent application Ser. No. 61/767,800 filed Feb. 22, 2013; the content of which is herein incorporated by reference in its entirety.

### TECHNICAL FIELD

The invention relates to a release device for releasing a medical implant from a catheter and to a catheter comprising a release device for releasing a medical implant for implantation in an animal and/or human body.

### BACKGROUND

In the field of medicine, implants are often used that are introduced into an animal and/or human body either permanently or at least for a relatively long period of time in order to carry out replacement functions. For example, these implants could include heart pacemakers, brain pacemakers for Parkinson's patients, cardiac implants, such as cardiac valves or what are known as septum-closure devices, cochlear implants, retinal implants, dental implants, implants for joint replacement, vessel prostheses, for example insertable into the pulmonary vein, occluders, for example for the appendix, or stents.

Implants are connected to catheters before insertion into the body and have to be able to be placed precisely at the site for use and released in a defined manner. To this end, it is known for example from U.S. Pat. No. 6,709,667 B1 to release the implant by a sliding motion.

### SUMMARY OF THE INVENTION

The object of the invention is to disclose a release device with which the intended release of an implant is improved.

A further object can be considered that of providing a corresponding insertion device.

The object is achieved in accordance with the invention by the features in the independent claims. Advantageous embodiments and advantages of the invention will emerge from the other claims and from the description.

A release device for releasing a medical implant from an insertion device is proposed, with which the implant can be released by a relative movement between a first and a second insertion element. The release device comprises a body with a proximal end, which is remote from a distal end of the insertion device during use, and with a distal end, which faces is the distal end of the insertion device during use, wherein a first grip segment, a second grip segment and at least one actuator are provided between the proximal and distal ends, wherein the at least one actuator is manually operable and can be brought substantially into the at least first grip segment substantially axially displaceably in order to effect an intended relative movement in the longitudinal direction between the first and second insertion element of the insertion device.

As a result of the embodiment according to the invention, a release device can be provided that can be operated intuitively and easily. It can also be held, manipulated and handled comfortably and securely by an operator, such as a doctor. In addition, a high release force can be introduced with low, in particular manual, operating force. Furthermore, powerful movements can be transferred by means of the actuator according to the invention, as a result of which the implant can be reliably and homogeneously released and also positioned precisely and carefully. The implant can thus be released powerfully and/or gradually and also carefully. In addition, such a simple concept for releasing the implant is implemented. The partially released implant can particularly advantageously be repositioned or withdrawn by means of the embodiments according to the invention, for example if positioned incorrectly. This is particularly the case since, in accordance with the device according to the invention, particularly large forces for again compressing the partially expanded implant can be produced. Furthermore, the release device can be easily handled and allows the implant to be easily assembled on the insertion device, for example a catheter, in the preparation laboratory. The complexity of the production process can also be reduced by the simple design, whereby production costs can also be kept low.

In this context, a “grip segment” is to be understood to mean a part of the release device that can be directly or indirectly engaged by one and/or more of the operator's hands so that the release device can be moved, manipulated and/or the like. Each of the grip segments can be formed by any element considered usable by a person skilled in the art, for example by the housing of the body, a part of the housing or by an additionally arranged grip, such as a U-shaped grip and a pistol grip. Furthermore an “actuator” is to be understood to be mean a structure or an element that directly and/or indirectly transfers an action performed by the operator onto at least part of the release device, for example one of the insertion elements, in particular to release or cover the implant. Hereinafter, the at least one actuator will also be referred to as the actuator.

The actuator can be formed by any element considered usable by a person skilled in the art, such as a pin, a bolt, a button, a rotary button, a push button, a slide or a lever. It is also advantageous if the actuator is formed by a control lever, whereby the actuator can be formed in a particularly robust and long-lasting manner. In addition, the actuator can be operated in a constructionally simple and comfortable manner from outside a housing of the body. With such an embodiment, large forces can be transferred in a user-friendly manner. In addition, compared to systems of the prior art, which operate with a small operating wheel, larger engagement areas can be provided. In addition, the force on the at least one insertion element or the outer shaft can be amplified via an individually presettable lever length of the control lever with use of the lever principle and, at the same time, the number of release steps can be determined (see below). In particular, an orientation or alignment of the actuator substantially perpendicular to a longitudinal axis of the body may constitute a preferred orientation or alignment of the actuator, whereby a space-saving arrangement and intuitive operation are possible.

Here, “manually operable” is to be understood to mean that the actuator can be grasped and/or operated immediately and directly, in particular manually, by the operator. Here, “substantially axially displaceably” means that a deviation of a displacement of the actuator of up to 10° from an axial direction is to be understood as axial displacement. Furthermore, the phrase “can be brought substantially into at least the first grip segment” is to be understood to mean that the actuator can be introduced and/or sunk into at least the first grip segment preferably by at least 50%, advantageously by at least 75% and particularly preferably by at least 95%. This is based in particular on an axial width of the actuator.

Here, an intended relative movement in the longitudinal direction in particular constitutes a movement in the axial and/or longitudinal direction. Here and in the following text, the term “effect” is to be understood to mean “produce, prompt and/or achieve”.

A starting position of the actuator is preferably a perpendicular arrangement of the actuator is relative to the direction of at least one of the insertion elements or an arrangement tilted at most by 5° to 10° relative to the perpendicular arrangement of the actuator. The term “tiltable” is to be understood as “pivotable” or “vibratable”. In addition, it would also be conceivable to provide a rotary and/or tilting movement of the actuator in its bearing point in addition to the substantially axial displacement of the actuator. The actuator can thus also be arranged inclined to the axial or longitudinal direction as a result of its movement. In addition, a substantially vertical movement of the actuator could also be advantageous. The definition of “substantially vertically” is to be understood similarly to the above-mentioned definition of “substantially axially”.

It is also advantageous if an axis of rotation of the first grip segment runs substantially perpendicularly to at least one of the insertion elements. With such an arrangement, a rotary movement of the first grip segment can be carried out substantially with no hindrance for the operator. In accordance with a preferred embodiment, the first grip segment may also run substantially perpendicularly to at least one of the insertion elements. The release device can thus be held tirelessly with a natural and relaxed hold for the operator. In this context, “substantially perpendicularly” is to be understood to mean a deviation from the direction of the axis of rotation or of the first grip segment relative to the direction of at least one of the insertion elements in relation to the perpendicular arrangement of up to 30°. In a preferred embodiment and as mentioned above, the first grip segment can be rotatable, and in particular is oriented about an axis of rotation substantially perpendicular to the longitudinal axis of the body. The first grip segment is most preferably formed by a pistol grip. A particularly ergonomic design can thus be provided, by means of which the first grip segment can be clasped by a whole hand. This is particularly user friendly since the first grip segment can be held tirelessly by the operator.

It is also proposed for the second grip segment to run substantially coaxially with at least one of the insertion elements, in particular the part of the at least one insertion element or of both insertion elements that is arranged within the body. A manipulation of the position of the release device can thus be transferred particularly reliably to at least one of the insertion elements. The term “substantially coaxially” is to be understood to mean an arrangement of the second grip segment that deviates at most by 5° to 10° from the axial arrangement of the grip segment. It is also proposed for the second grip segment to be formed integrally with a housing of the body or the release device, whereby a large engagement area for handling the release device is created. Here, “integrally” is to be understood to mean that the second grip segment and the housing are formed by the same component and/or can only be separated from one another with a loss of function of at least one of the components.

The movement of the actuator can be transferred to at least one of the insertion elements or the outer insertion element by means of any concept that appears to be appropriate to a person skilled in the art, for example by an integral bond between the actuator and at least one of the insertion elements or the outer insertion element or by an interlocking and/or force-locked connection between the actuator and one of the insertion elements or the outer insertion element or a further element and/or component connectable to the insertion element, such as a transfer element. In the latter case, the connection between the actuator and one of the insertion elements is an indirect connection or a connection conveyed by the further element and/or component.

With the present invention, it can be assumed that there is enough friction between the outer insertion element and the body so that the outer insertion element remains fixed in its position as the actuator and/or the transfer element is moved back by a spring element (see below) or does not move with the actuator and/or the transfer element. The intended relative movement in the longitudinal direction between the first and the second insertion element can thus advantageously be carried out in small release steps. This is achieved by actuating the actuator a number of times successively.

In accordance with a preferred embodiment, the intended relative movement in the longitudinal direction between the first and the second insertion element of the insertion device is achieved at least by means of a force-locked connection between the actuator and one of the insertion elements. As a result of the force-locked connection, a proven and robust principle of force transfer can be used. In this case, the phrase “by means of a force-locked connection between the actuator and one of the insertion elements” is to be understood to mean both a direct force-locked connection and an indirect force-locked connection, conveyed via the further element and/or component and/or the transfer element between the actuator and one of the insertion elements. An element that transfers the forward movement via a force-locked connection onto one of the insertion elements, such as the actuator or a transfer element in particular, is formed from at least one very hard and/or rigid material, for example Inox AISI 316L or Inox AISI 306. The insertion element is preferably the outer insertion element. The force-locked connection can be achieved in a constructionally simple manner by the actuator and/or by an axially and/or vertically offset and/or tilted position of the actuator and can be transferred to the at least one insertion element or the outer shaft.

The intended relative movement in the longitudinal direction between the first and the second insertion element of the insertion device is preferably achieved at least by means of a releasable force-locked connection between the actuator and one of the insertion elements or the outer insertion element. The stage-like or gradual release of the implant can advantageously be implemented easily hereby.

Alternatively and/or additionally, the intended relative movement in the longitudinal direction between the first and the second insertion element of the insertion device is achieved at least by means of an interlocking connection between the actuator and one of the insertion elements. To this end, a material of the at least one insertion element may preferably be adapted accordingly and for example comprises a material having high static friction. The at least one insertion element can thus be held in position relative to the actuator in the tilted state. The material can be any material having a high static friction considered suitable by a person skilled in the art.

It is also proposed for the release device to have a control element, with which an operating mode can be set, whereby the mode can be easily changed. The control element can be formed by any element considered applicable by a person skilled in the art, such as a push button, a lever, a rotary button or a control slide. It may also be advantageous if the release device has a control element, with which an operating mode can be displayed. Two functions can thus be combined in one component in a space-saving and cost-saving manner and with simpler assembly. It is additionally proposed for the release device to have a display element for displaying a set operating mode. The display element can be formed by any element considered appropriate by a person skilled in the art, such as a display, a scaling, a text field or a component orientation. A display element is also to be understood to mean a unit of a plurality of identical or different display elements. The control element and the display element are advantageously formed in one piece, whereby a compact device can be achieved. Here, “in one piece” is to be understood to mean that the control element and the display element are formed by the same component and/or can only be separated from one another with the loss of function of at least one of the components.

In accordance with an advantageous embodiment, the control element may have an active element for changing between at least two operating modes, whereby the operating mode can be easily determined. The active element can be formed by any element considered applicable by a person skilled in the art, such as a spring, a lever, a sliding switch or in particular an eccentric element. An effect of the active element can be obtained particularly easily if it is arranged eccentrically to an axis of rotation of the control element. In accordance with the embodiment according to the invention, the actuation of the control element, in particular the rotation thereof in the embodiment as a rotatable grip segment, can convey in a constructionally simple manner different reactions to different components or constellations of different components, for example to the actuator or a recess in the first grip segment or the arrangement thereof relative to one another. Here, the axis of rotation is aligned in particular substantially perpendicularly to the longitudinal direction of the at least one insertion element and an extension of the actuator. The active element is preferably connected in one piece to the control element or is formed therein. The definition of “in one piece” is to be understood similarly to the definition as described above.

The first grip segment is advantageously formed in one piece with the control element, whereby the operation of the control element can be triggered in a process-streamlining manner and without delay. In accordance with an advantageous embodiment, the display element is preferably formed in one piece with a rotatable grip segment. Space can thus be saved advantageously. The rotatable grip segment is preferably the first grip segment. The definition of “in one piece” is to be understood in each case similarly to the definition described above. If the rotatable grip segment, the display element and the control element are combined in one component, the operator can set and also detect an operating mode in a particularly comfortable and quick manner. The rotatable grip segment is preferably formed by an asymmetrical component, wherein the operating mode and preferably also direction of movement, for example of the outer insertion element, can be indicated by the orientation of the grip segment, in particular in a manner that is intuitive for the operator. It is also proposed for the release device to comprise at least one substantially axially displaceable transfer element, whereby the movement of the actuator can be transferred in a constructionally simple manner. In addition, the different components, such as the actuator and the transfer element, can thus advantageously be fabricated from different materials, which are specifically matched to a function or a load of the component. Reference is made to the definition above with regard to the definition of “substantially axially displaceably”. The “substantially axially displaceable transfer element” will be referred to hereinafter as “the transfer element”. For fast and reliable transfer, the transfer element transfers the axial movement of the actuator, advantageously upon actuation of the actuator, to at least one of the insertion elements and in particular the outer insertion element in order to effect the intended relative movement in the longitudinal direction between the first and the second insertion element of the insertion device.

It may also be advantageous for the transfer element to be formed in one piece with the outer insertion element. The connection of these components can thus be ensured captively and reliably.

In accordance with an alternative and preferred development, the transfer element is formed as a component separate from the outer insertion element, whereby, in this case to too, materials of the components can be designed individually on the basis of the function of said components, etc. Here, the transfer of the movement of the actuator to the transfer element can advantageously be conveyed by means of a releasable connection. In this case too, this can be achieved by means of any concept that appears to be applicable to a person skilled in the art, for example an interlocking connection, a force-locked connection and/or a frictional connection.

Any connection type considered suitable by a person skilled in the art, for example any above-mentioned connection type, would also be conceivable for a connection between the actuator and the transfer element. Accordingly, the actuator may be arranged on/in the transfer element with a force-locked and/or interlocking connection for example. It would also be conceivable for the actuator and/or the transfer element to be provided with a coating for example that conveys a high static friction. A material of such a coating can be any material considered appropriate by a person skilled in the art, such as a polymer in particular, and in particular a material selected from the group consisting of polyamide, polyester, polyether block amide, silicone and polyurethane. The clamping body can hereby be designed in particular with low weight. Particularly reliable positioning of the implant in the insertion device can advantageously be achieved due to its high static friction, for example if the material is a polyether block amide, such as PEBAX, from Arkema. All degrees of hardness can be used in this case.

In a further impossible embodiment, the transfer element has at least one first stop for the actuator. A movement of the actuator can thus be easily transferred to the transfer element and one of the insertion elements or the outer insertion element. The transfer element preferably has at least one second stop for the actuator, wherein the first and the at least second stop are preferably arranged axially on different sides of the actuator, whereby these can limit or transfer opposed movements of the actuator.

Here, the transfer element can be any element considered appropriate by a person skilled in the art, such as a pin, a bolt, a plate, a disc, a ring or a sleeve. In a preferred embodiment, the at least one transfer element has a cylindrical sleeve, whereby a robust and reliable component can be used. The transfer element is therefore also matched to a shape of the to insertion elements and in particular of the outer insertion element. A space-saving arrangement can be provided if the sleeve is arranged substantially coaxially with at least one of the insertion elements or the outer insertion element.

In addition, it may be advantageous for the transfer element to have at least one passage for at least one of the insertion elements and in particular an outer insertion element and/or an outer shaft. This allows a compact arrangement and a constructionally simple option for achieving an effective connection between the transfer element and at least one of the insertion elements. In this context, an effective connection is to be understood in particular to mean an interlocking connection and/or a force-locked connection.

For example, the transfer element can also be formed such that the substantially axial movement of the actuator triggers a tilting movement of the transfer element. Said transfer element is thus canted on the outer insertion element and the outer insertion element can be axially moved in a constructionally simple manner with the transfer element as a result of this force-locked connection or this clamping effect. In this case, the transfer element could be formed for example as a sleeve widening conically in the direction of movement. If, in addition to the axial movement of the actuator, a pivoting or a tilting movement of the actuator is provided, a possible tilting of the transfer element can be implemented easily, for example by a stationary engagement of the actuator with the transfer element. With a tiltable embodiment of the transfer element and/or of the actuator, the coaxial position of the transfer element and/or a perpendicular position of the actuator with respect to at least one of the insertion elements constitutes a contactless position, whereas the tilted or inclined position constitutes a contact position.

In principle, the transfer element could also be embodied however such that the radial diameter thereof conveyed by the axial displacement of the actuator reduces and the insertion element is thus pinched for example. In terms of construction, this could be implemented for example by an additional vertical movement of the actuator.

In accordance with a further embodiment, the transfer element has at least one force transfer region, whereby this region can be adapted, with regard to its dimension(s), its material, its shape, etc. for example, specifically to its function of the force transfer of the force-locked connection between the actuator and one of the insertion elements. In particular, the force transfer region can be brought into contact with the actuator or is constantly in contact therewith.

The transfer element also has at least one sliding region, wherein the at least one sliding region preferably conveys a sliding movement of the transfer element to one of the insertion elements. The transfer element can thus move relative to the (outer) insertion element in a particularly homogeneous manner and with reduced friction, which is in particular advantageous in a mode of fast release of the implant. Here, it would be conceivable either to equip a complete part of the transfer element from a material having good sliding properties or also to apply a coating made of such a material to only the sliding region. Any material considered usable by a person skilled in the art would be conceivable as such a material, for example Teflon, graphite or the like. In a preferred embodiment, the force transfer region is flanked axially by two sliding regions, whereby a sliding movement can be carried out particularly uniformly. These two sliding regions can be embodied identically or may differ in terms of at least one parameter, such as dimension (axial length, radial thickness), contour, shape, material, coating, etc.

In accordance with a preferred and particularly advantageous embodiment of the invention, the transfer element comprises at least one transition region, whereby this region can be provided for specific functions. The transition region is preferably arranged axially between the at least one force transfer region and the at least one sliding region. These regions may thus be formed independently of one another in terms of their properties. The transition region may also have its own specific embodiment however and may thus perform a specific function. For example, the transition region and the force transfer region and/or the sliding region may each have a radial material thickness, wherein in particular the material thickness of the transition region is thinner than the material thickness of the force transfer region and/or of the sliding region. The transition region thus has reduced flexural rigidity compared to the force transfer region and/or the sliding region. It may thus be possible for example for the force transfer region of the transfer element to assume a to different orientation or position, such as a tilted or canted or vertical position, compared to the sliding region of the transfer element. In principle, this could also be ensured by another or softer material of the transition region compared to the force transfer region and/or the sliding region.

In a further embodiment of the invention, it is proposed for the transfer element to be biased by at least one spring element, whereby the transfer element can be held in a set position in a constructionally simple manner. Here, a “spring element” is to be understood to mean any resilient and/or elastic element, and in particular a spring, for example in the form of a compression spring. It is also proposed for the transfer element to be able to be returned into its starting position by means of the spring element. As a result of this advantageous embodiment, an additional restoring means, operable by an operator, for the spring element and/or the transfer element to be omitted, whereby assembly complexity, installation space and costs can be saved. In addition, with the preferred embodiment of the spring element as a return spring, a reliable component having a low weight can be used. In this case, it is assumed that there is enough friction between the outer insertion element and the body so that the outer insertion element remains fixed in its position as the at least one transfer element is moved back by the spring element or does not move with the at least one transfer element. In order to prevent this in any case, a blocking element may additionally be provided, said blocking element holding the outer insertion element in position. This blocking element can be formed by any element considered usable by a person skilled in the art, such as an area of high friction or a blocking disc, which for example is placed radially around the outer insertion element.

A release device that can be used in a versatile and flexible manner can advantageously be provided if an operating mode of fast release of the implant is provided. This operating mode can be set in a constructionally simple manner if, for the fast release of the implant, the transfer element is substantially coaxially adjustable relative to at least one of the insertion elements and/or is held in a coaxial position. Reference is made to the embodiments above with regard to the definition of “substantially coaxially”. In the coaxial arrangement the force-locked connection between the actuator and/or the transfer element and the at least one insertion element or the outer shaft is removed or there is no force-locked connection between the actuator and/or the transfer element and the at least one insertion element or the outer shaft.

The axial direction of movement of the actuator advantageously has a same direction as the movement of the outer insertion element, whereby the operation of the actuator is particularly intuitively and easily understandable for the operator. Alternatively, it may also be the same direction. It is also proposed for an axial movement of the actuator in the direction of the proximal end of the body to convey a release of the implant. The active mechanism is thus intuitively understandable for the operator. In a further embodiment of the invention, it is proposed for an axial movement of the actuator in the direction of the distal end of the body to convey a covering of the implant with one of the insertion elements, whereby this movement conveyance can also be easily comprehended by the user. By means of the different directions for the release and the covering of the implant, the two main functions of the release device can be conveyed with reduced error. Here, a covering of the implant is understood to mean a shrouding of the implant with the outer insertion element in particular.

In accordance with a preferred development, the body has at least one receptacle for an inner insertion element for immovably fixing the inner insertion element to the body. The inner insertion element or an inner shaft can thus be held or anchored securely on the body. The receptacle can be formed by any structure considered appropriate by a person skilled in the art, such as a hook, a bolt, a recess, an indentation, a gap or a slit. In addition, it is proposed for the receptacle to be formed on the outer insertion element or on the outer shaft, whereby the anchoring is designed particularly directly and immediately. For this purpose, the outer insertion element or the outer shaft has to be designed such that it can take up tensile and compressive forces without buckling. A particularly good fixing can advantageously be achieved if the receptacle is embodied as a slit. Alternatively and/or additionally, the inner outer shaft is fixed in a further anchoring on the body and/or housing thereof. The outer insertion element can thus be relieved of load. For this purpose, any connection type considered appropriate by a person skilled in the art, such as a force-locked connection, an interlocking connection or an integral bond, for example by means of welding, soldering, screwing, nailing, crimping or adhesive bonding, can be considered.

In a further embodiment of the invention, it is proposed for the body to have at least one passage for at least one of the insertion elements. This allows a compact arrangement, which stabilizes and protects the insertion element passed through. If the insertion device is a catheter, the insertion element in question may be an outer insertion element of the catheter.

In accordance with an additional embodiment, it is proposed for an insertion element protruding from the body, in particular such as the outer insertion element protruding from the body at the proximal end thereof, to have at least one marking, which is intended to indicate the extent to which the implant has been released. A progression of the release of the implant can thus be monitored in a particularly simple manner. Such a marking can be formed by any means considered appropriate by a person skilled in the art, for example a number, a letter, a line, a notch, a color code or the like. A multiplicity of markings is advantageously provided. It is additionally advantageous if the marking is intended to indicate whether the implant can be retracted into the release device. The implant can thus advantageously be prevented from becoming blocked in the release device, and a malfunction of the release device can thus be avoided.

In accordance with a further aspect of the invention, an insertion device for insertion of the medical implant, which can be released by a relative movement between the first and the second insertion element, is proposed and comprises the release device for releasing the medical implant, said release device comprising the body with the proximal end, which is remote from the distal end of the insertion device during use, and with the distal end, which faces the distal end of the insertion device during use, wherein a first grip segment, a second grip segment and at least one actuator are provided between the proximal and the distal end, wherein the at least one actuator is manually operable and can be brought substantially into at least the first grip segment substantially axially displaceably in order to effect an intended relative movement in the longitudinal direction between the first and the second insertion element of the insertion device.

As a result of the embodiment according to the invention, an insertion device can be provided that can be operated intuitively and easily. It can also be held, manipulated and handled comfortably and securely by an operator, such as a doctor. In addition, a high release force can be introduced with low, in particular manual, operating force, whereby, in addition, powerful movements can be transferred, as a result of which the implant can be reliably and homogeneously released and also positioned precisely and carefully. The implant can thus be released powerfully and/or gradually and also carefully. The partially released implant can particularly advantageously be repositioned or withdrawn by means of the embodiments according to the invention, for example if positioned incorrectly. Furthermore, the insertion device can be easily handled and allows the implant to be easily assembled in the preparation laboratory. The complexity of the production process can also be reduced by the simple design, whereby production costs can also be kept low.

In accordance with an advantageous embodiment, the implant may be a self-expanding implant, whereby it can open automatically upon release. Due to the self-expanding implant, an additional expanding means can be omitted. Space and assembly effort for this can therefore be saved. The insertion device can thus also be formed in a less complex manner. In principle however, it would also be possible to use a balloon-expandable implant. To this end, the insertion device would have to be adapted accordingly however, which a person skilled in the art achieves independently on the basis of his common general knowledge in the art.

### DESCRIPTION OF THE DRAWINGS

The invention will be explained in greater detail hereinafter by way of example with reference to exemplary embodiments illustrated in drawings, in which:

FIG. 1 shows a schematic illustration of a section through a favorable exemplary embodiment of an insertion device and a release device with an assembled implant;

FIG. 2 shows a schematic illustration of a section through the release device in FIG. 1 along line II-II;

FIG. 3A shows a schematic illustration of the release device in FIG. 1 prepared for release of the implant;

FIG. 3B shows a schematic illustration of a side view of a grip segment of the release device in FIG. 1 with display of the operating mode for slow release of the implant;

FIG. 4 shows a schematic illustration of the release device in FIG. 1 during the slow release of the implant;

FIG. 5A shows a schematic illustration of the release device in FIG. 1 prepared for a covering of the implant;

FIG. 5B shows a schematic illustration of a side view of the grip segment of the release device in FIG. 1 with display of the operating mode for covering the implant;

FIG. 6 shows a schematic illustration of the release device in FIG. 1 as the implant is covered;

FIG. 7A shows a schematic illustration of the release device in FIG. 1 prepared for a fast release of the implant;

FIG. 7B shows a schematic illustration of a side view of the grip segment of the release device in FIG. 1 with display of the operating mode for fast release of the implant;

FIG. 8 shows a schematic illustration of an alternative release device prepared for a slow release of an implant;

FIG. 9 shows a schematic illustration of the release device in FIG. 8 during the slow release of the implant;

FIG. 10 shows a schematic illustration of the release device in FIG. 8 prepared for covering of the implant;

FIG. 11 shows a schematic illustration of the release device in FIG. 8 as the implant is covered, and;

FIG. 12 shows a schematic illustration of a further alternative release device during the slow release of the implant.

### DETAILED DESCRIPTION

In the figures, functionally like or similarly acting elements are denoted in each case by like reference signs. The figures are schematic illustrations of the invention. They do not show specific parameters of the invention. The figures also merely reproduce typical embodiments of the invention and are not intended to limit the invention to the embodiments illustrated.

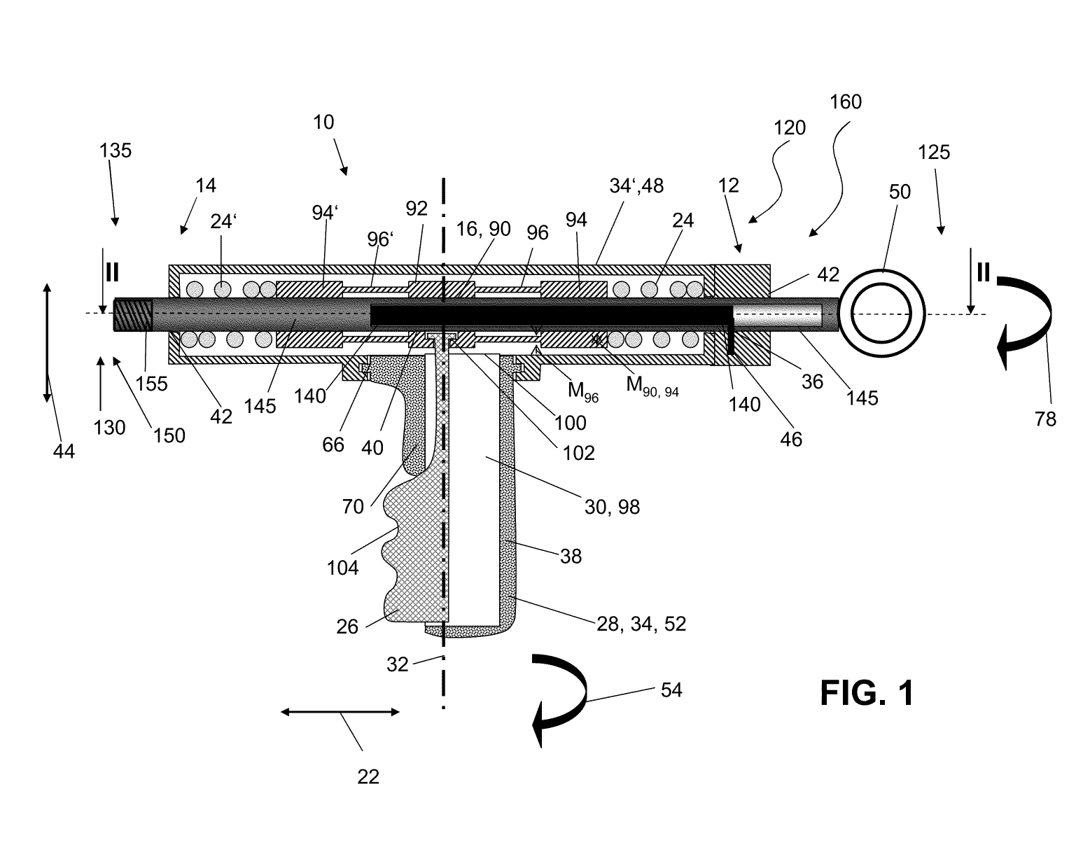

FIG. 1 shows a longitudinal section through a favorable exemplary embodiment of a release device 160 of an insertion device 120. For example, the insertion device 120 is a catheter having a shaft region 135 with two coaxially arranged insertion elements 140, 145, for example an inner shaft (insertion element 140) and an outer shaft (insertion element 145), which surrounds said inner shaft and in turn can be surrounded by an outer sleeve (not illustrated here). The proximal end 125 of the insertion device 120 faces an operator during use, that is to say as the implant 155 is fastened to the release device 160 or during implantation. The implant 155 is placed at the distal end 130 of the shaft region 135 between the inner shaft and the outer shaft and is to be released at the site of implantation in the animal or human body.

The release device 160 is used to release the medical implant 155 from the insertion device 120. The implant 155 is arranged at the end 130 of the shaft region 135 remote from the operator, for example in the vicinity of a catheter tip 150 (not shown here in detail). For example, the implant 155 is placed around the inner insertion element 140 (not shown in detail) and is released by a relative movement between the first and the second insertion is element 140, 145. In this case, the inner insertion element 140 is connected to the catheter tip 150, but by contrast the outer insertion element 145 is not.

The release device 160 comprises a body 10 with a proximal end 12, which is remote from the distal end 130 of the insertion device 120 during use, and with a distal end 14, which faces the distal end 130 of the insertion device 120 during use. The body 10 or the outer insertion element 145 has a receptacle 36, in the form of an axial slit, for a proximal end of the inner insertion element 140 in order to immovably fix the inner insertion element 140 to the body 10. In addition, the proximal end of the inner insertion element 140 is held in the radial direction 44 after the receptacle 36 of the outer insertion element 145 in an anchoring 46 of a housing 48 of the body 10, for example with an interlocking connection, force-locked connection or integrally bonded connection. In addition, the body 10 has a plurality of passages 42 for the outer insertion element 145, whereby said outer insertion element exits from the body 10 at the proximal end 12 thereof. An operating element 50 in the form of a loop grip is formed at a proximal end of the outer insertion element 145 outside the body 10.

The release device 160 or the body 10 has a first grip segment 34 and a second grip segment 34′ between the proximal and distal ends 12, 14. The first grip segment 34 is arranged in an axially central position of the body 10 and runs substantially perpendicularly to two insertion elements 140, 145 or is aligned substantially perpendicularly to the body 10. In a functioning situation, for example during implantation, the first grip segment 34 points downwardly relative to the body 10. In addition, the first grip segment 34 is formed as a pistol grip 52. The first grip segment 34 is also embodied, or mounted in the housing 48 via an axially protruding disc 66, such that it can be rotated about its axis of rotation 32 in the peripheral direction 54 of the first grip segment 34. The axis of rotation 32 of the first grip segment 34 runs substantially perpendicularly to the insertion elements 140, 145. By contrast, the second grip segment 34′ is formed in one piece with the housing 48 and thus runs substantially coaxially to both insertion elements 140, 145.

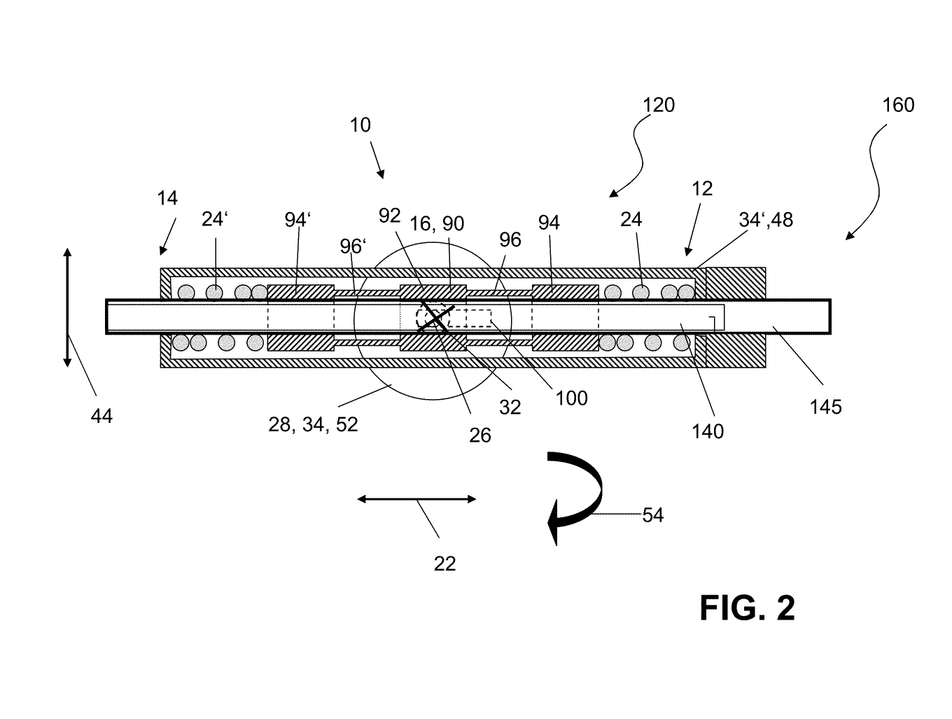

In addition, an actuator 26 is arranged on the first grip segment 34, is manually operable by the operator and can be brought into the first grip segment 34 substantially axially displaceably in order to effect an intended relative movement in the longitudinal direction 22 between the first and second insertion element 140, 145 of the insertion device 120 (see below for details). The actuator 26 is formed as a control lever. In addition, in its unactuated state the actuator 26 is arranged partly in a recess 98 in the first grip segment 34 and extends substantially perpendicularly to the axis of rotation 32 of the first grip segment 34. An operating surface 104 or an engagement face of the actuator 26 is arranged outside the recess 98. Furthermore, the actuator 26 extends through an axially extending slit 100 in the housing 48 of the body 10 (see FIG. 2) and engages in a receptacle 102, which is formed in a transfer element 16.

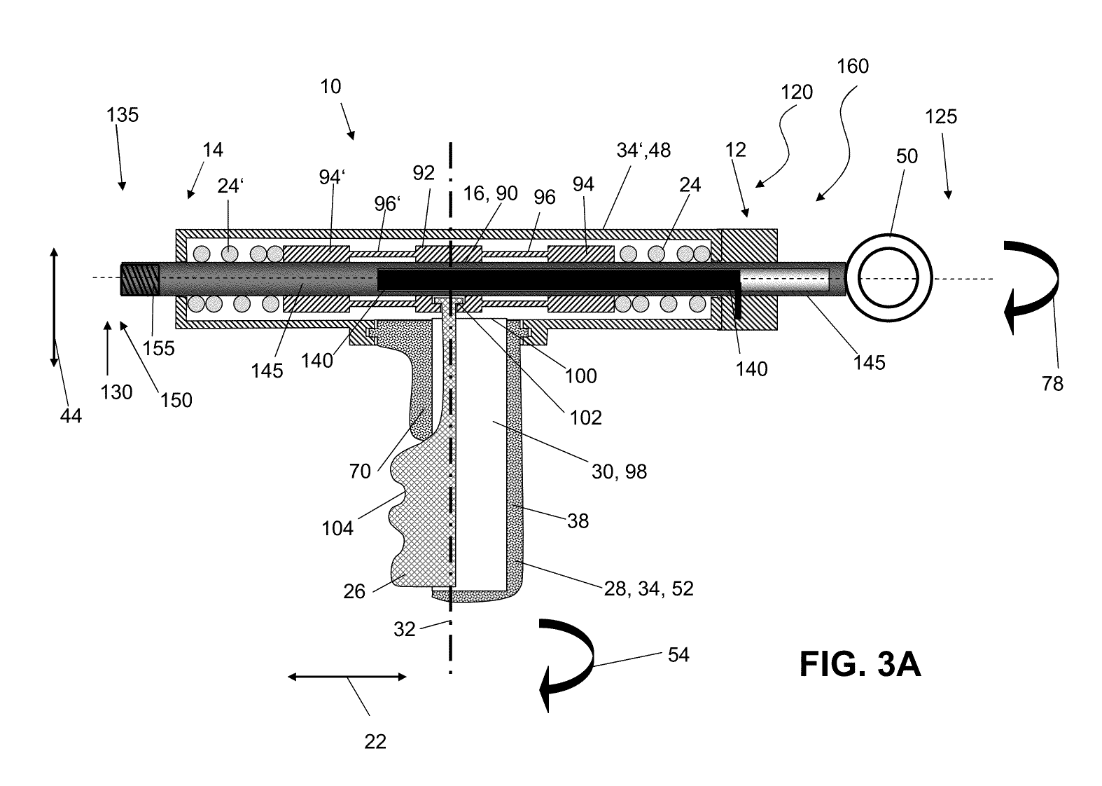

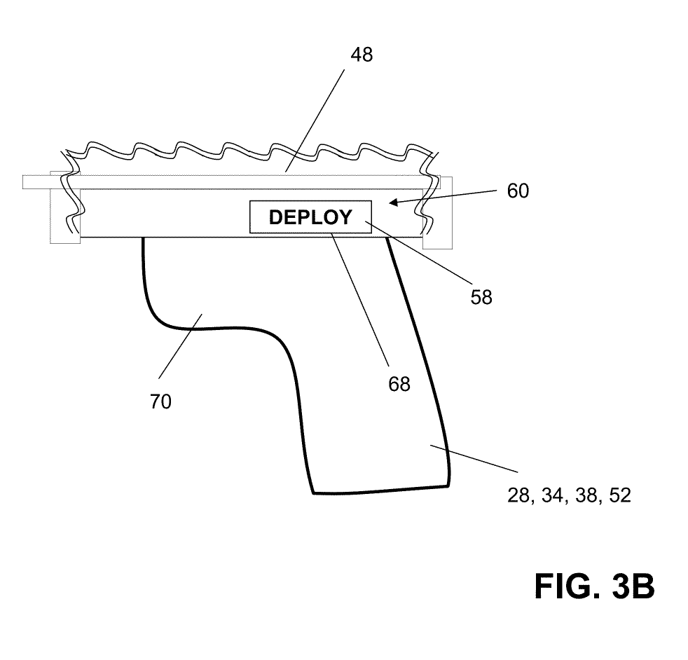

The release device 160 also has a control element 28, with which an operating mode of the release device 160 can be set (see below). Here, the control element 28 is formed in one piece with the first grip segment 34 or the pistol grip 52. In addition, the release device 160 has a display element 38 for displaying the set operating mode. The display element 38 is also formed in one piece with the rotatable grip segment 34 or the pistol grip 52 and, depending on the operating mode, is formed by lettering 58 indicating the operating mode, said lettering being visible in a display 60 of the housing 48 of the body 10 depending on the operating mode set (see FIGS. 3B, 5B and 7B).

Between the proximal and distal end 12, 14 of the body 10, said body has the transfer element 16, which is substantially axially displaceable and transfers the axial movement of the actuator 26 upon actuation of the actuator 26 to the outer insertion element 145 in order to effect the intended relative movement in the longitudinal direction 22 between the first and the second insertion element 140, 145 of the insertion device 120. The transfer element 16 is formed as a force transfer region 90 of a cylindrical sleeve 92, which is arranged substantially coaxially with both insertion elements 140, 145 or extends in a peripheral direction 78 of the body 10 along a periphery of the outer insertion element 145. The transfer element 16 thus has a passage 40 for the outer insertion element 145. The force transfer region 90 for transferring the movement of the actuator 26 also comprises the receptacle 102. The sleeve 92 is captively connected to the outer insertion element 145 in order to is transfer the movement of the actuator 26. In this case, any force-locked connection, interlocking connection and/or integrally bonded connection can be considered between the sleeve 92 and the outer insertion element 145.

The outer insertion element 145 and the force transfer region 90 and therefore the actuator 26 are preferably connected via a releasable force-locked connection. For this purpose, the release device 160 has a means for producing and releasing the force-locked connection, for example a clamping means (not shown here in greater detail). This is used with a gradual release or covering of the implant 155 (see below).

The cylindrical sleeve 92 additionally has two end regions 94, 94′, wherein one of the end regions 94 is arranged at an end of the sleeve 92 pointing in the direction of the proximal end 12 of the body and the other end region 94′ is arranged at an end of a sleeve 92 pointing in the direction of the distal end 14 of the body. Both end regions 94, 94′ are connected to the force transfer region 90 via a transition region 96, 96′. The force transfer region 90 and the end regions 94, 94′ each have substantially the same material thickness M₉₀, M₉₄ in the radial direction 44 of the sleeve 92. By contrast, the transition regions 96, 96′ have a radial material thickness M₉₆, which is thinner than the material thicknesses M₉₀, M₉₄ of the force transfer region 90 and of the end regions 94, 94′.

The transfer element 16 is biased by two spring elements 24, 24′, which are both formed as a compression spring. The spring element 24 extends between the proximal end 12 of the body 10 and the end region 94 of the transfer element 16, and the other spring element 24′ extends between the distal end 14 of the body 10 and the end 94′ of the transfer element 16. In a starting configuration set for example before implantation, the transfer element 16 is biased by each spring element 24, 24′ and is axially fixed between the spring elements 24, 24′ in an axially central position in the housing 48 of the body 10.

Two different operating modes can be conveyed by means of the actuator 26, more specifically a release of the implant 155 and a covering of the implant 155 by the outer insertion element 145. The control element 28 has an active element 30 in order to change between the two operating modes. This active element 30 is formed by the recess 98 in the first grip segment 34 and is arranged eccentrically relative to the axis of rotation 32 of the control element 28. The actuator 26 is thus also arranged eccentrically in the recess 98. In the unactuated state of the actuator 26, the transfer element 16 or the sleeve 92 is, in this position, in its axially central position, in which both spring elements 24, 24′ are equally biased.

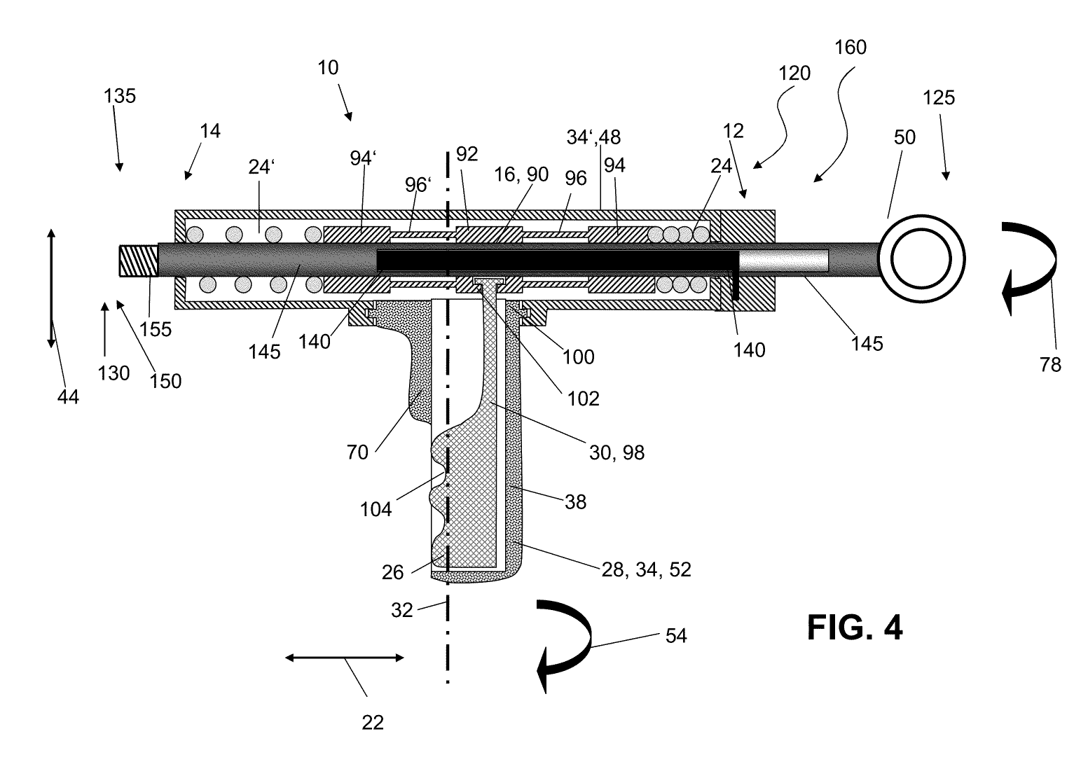

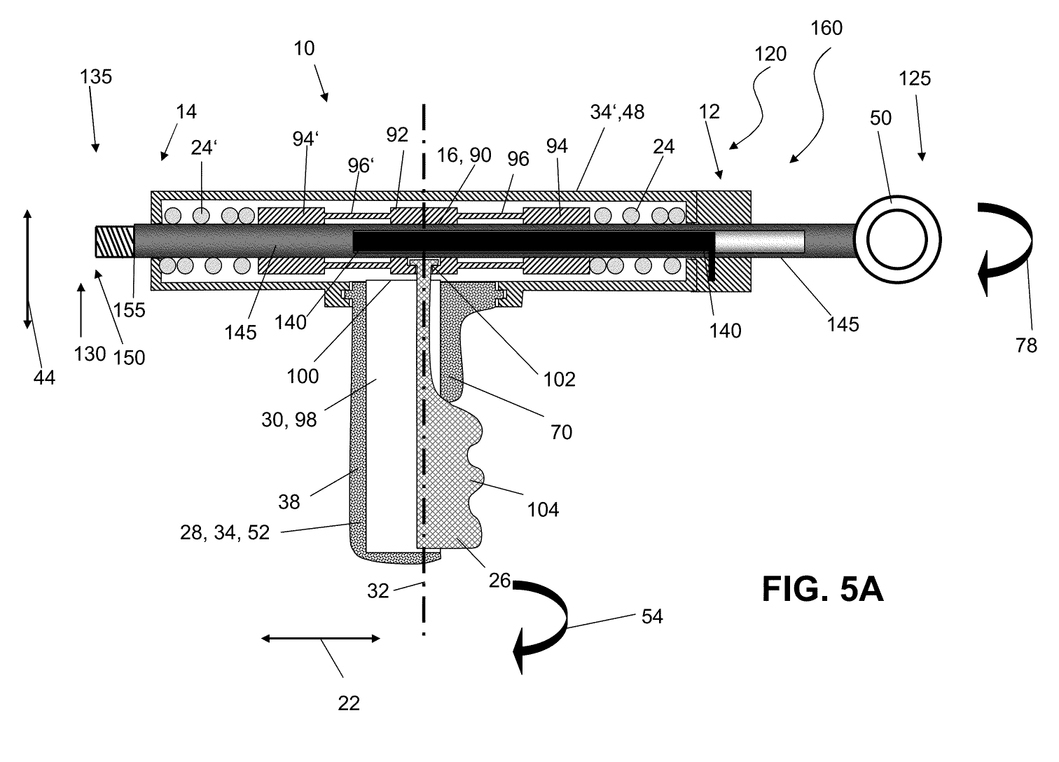

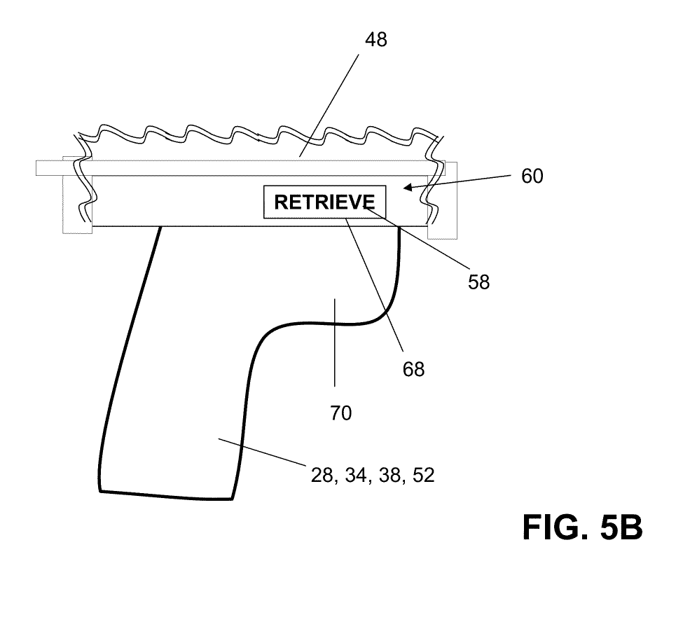

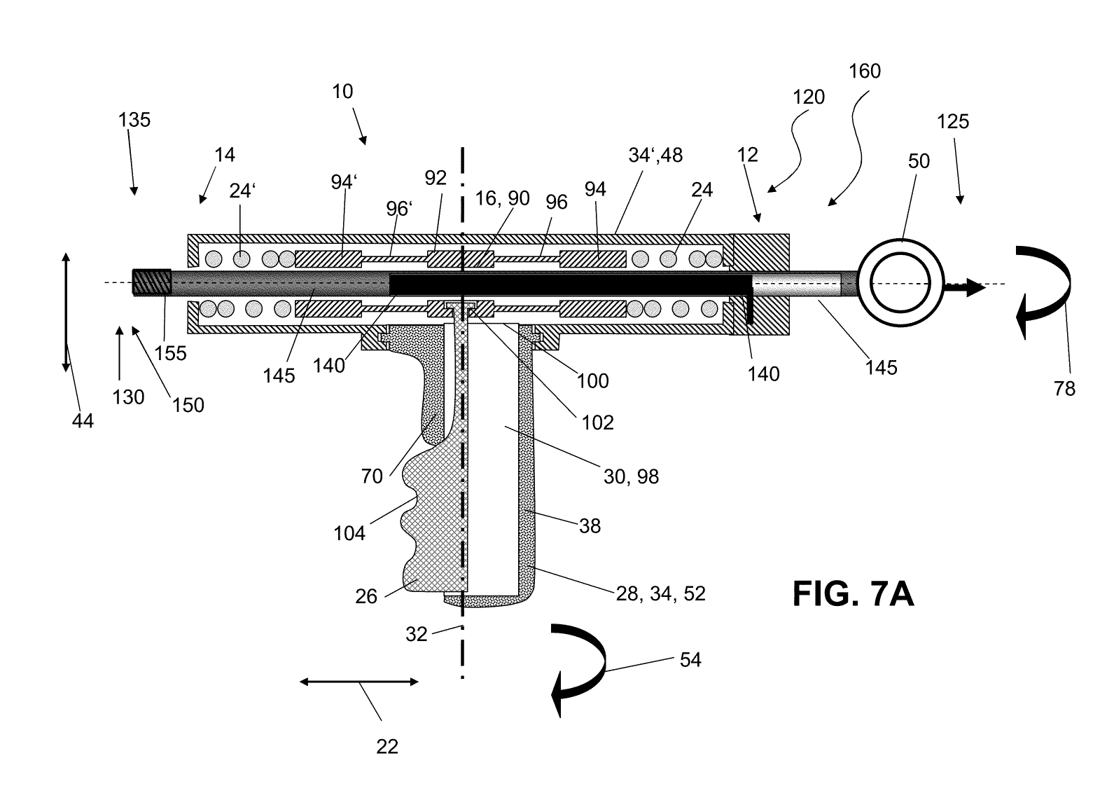

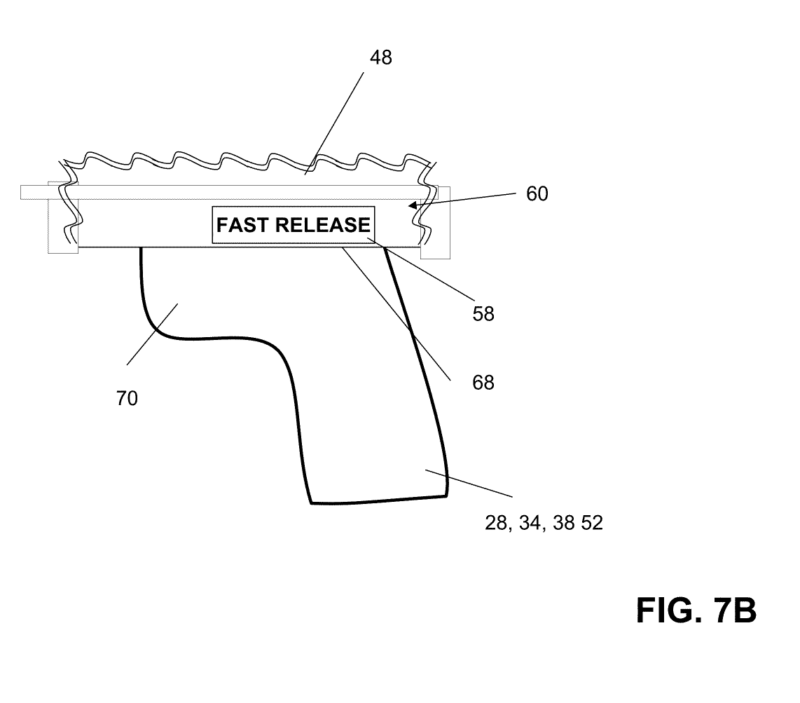

A slow release of the implant 155 on the basis of what is known as a deploy mode will now be described on the basis of FIGS. 3A, 3B and 4. To implant the implant 155 in the body, the insertion device 120 thus prepared is introduced into the body (not shown). To release the implant 155, the control element 28 or the first grip segment 34 (pistol grip 52) is rotated in the peripheral direction 54 until the lettering 58 “DEPLOY” appears in the display 60 (see FIG. 3B, the actuator 26 is not shown here, nor in FIG. 5B or 7B). This may also be the preset operating mode. The lettering 58 DEPLOY can be engraved for example in a wheel (not illustrated here in greater detail), which rotatably mounts the first grip segment 34. A window 68 in the housing 48 allows the corresponding lettering 58 on the wheel to appear depending on the position of the grip segment. The pistol grip 52 is also formed as a display element 38 since it indirectly displays the direction of movement of the outer insertion element 145 by orienting its pommel 70 against an index finger (not shown here). This means that, in order to release the implant 155, the outer shaft will be moved in the direction of the proximal end 12 and the pommel 70 of the first grip segment 34 will point in the direction of the distal end 14. The operating surface 104 of the actuator 26 also points in the direction of the distal end 14. The eccentric active element 30 or the recess 98 is arranged here such that a clearance for sinking the actuator 26 is provided in the direction of the proximal end 12. In this setting, the sleeve 92 is in its central position in the body 10 and the spring elements 24, 24′ are substantially hardly compressed.

In this case, the force transfer region 90 and the outer insertion element 145 are interconnected with a force-locked connection. The clamping means is operated in order to produce the force-locked connection. For example, said clamping means is formed as a compression ring or a press switch, which for example reduces a diameter of the force transfer region 90 or changes the round contour thereof to an unround contour (not shown in detail). With an embodiment as a press switch, this would enter the housing 48 upon actuation by is the operator from outside the body 10 and can radially contact the transfer region 90 upon actuation. In addition, it preferably moves with the transfer region 90 in the event of the movement in the longitudinal direction 22 and in doing so is guided by an axially extending slit in the housing 48. In a constructionally simple embodiment, the press switch would be arranged radially opposite the actuator 26 for example and would press onto the portion of the transfer element 16 arranged radially opposite the receptacle 102 of the actuator 26. Due to the lower material thickness M₉₆ of the transition regions 96, 96′ and therefore the lower flexural rigidity thereof compared to the material thicknesses M_{90, 94} of the force transfer region 90 and the end regions 94, 94′, the force-locked connection is only produced between the force transfer region 90 of the sleeve 92 and the outer insertion element 145 and not between the end regions 94, 94′ of the sleeve 92 and the outer insertion element 145.

Upon actuation of the actuator 26 or the control lever in this operating mode, it is displaced axially in the direction of the proximal end 12. It thus moves completely into the recess 98 in the first grip segment 34 (see FIG. 4). Due to the engagement of the actuator 26 with the receptacle 102 of the force transfer region 90, the force transfer region 90 is also moved parallel to the longitudinal direction 22 in the direction of the proximal end 12. Due to the captive connection between the sleeve 92 and the outer insertion element 145, said insertion element is also slid in the direction of the proximal end 12. This occurs until the spring element 24 is fully compressed, which limits the movement of the actuator 26.

To move the transfer element 16 into its starting position, the force-locked connection between said transfer element and the outer insertion element 145 then has to be released. This is achieved by releasing the clamping means. If the actuator 26 is then disengaged, the spring element 24, which is also a return spring, can expand and presses the sleeve 92 with the force transfer region 90 and the actuator 26 in the longitudinal direction 22 and in the direction of the distal end 14 back into the central position or the starting position (see FIG. 3). The end regions 94, 94′ of the sleeve 92 are formed as sliding regions 94, 94′ in order to convey a sliding movement of the transfer element 16 to the outer insertion element 145. Since, with the present invention, it can be assumed that there is much friction between the outer insertion element 145 and the body 10, the outer insertion element 145 is remains fixed in its position as the actuator 26 and the transfer element 16 are moved back or it does not move back with the actuator 26 and the transfer element 16. The process of releasing the implant 155 can thus be repeated until the outer insertion element 145 has completely released the implant 155. The extent to which the implant 155 has been released can be monitored for example by a marking (not illustrated here in greater detail). This marking is applied for example to an end of the outer insertion element 145, which protrudes from the body 10 at the proximal end 12 thereof. The marking can be formed as a series of ascending numbers for example.

By means of its axial movement, the actuator 26 thus conveys the intended relative movement in the longitudinal direction 22 between the first and the second insertion element 140, 145, or, as a result of the actuation of the actuator 26, the outer shaft is withdrawn relative to the inner shaft and therefore also to the implant 155. The implant 155 is thus exposed and expands automatically for example with the embodiment as a self-expandable implant 155, such as a stent-based cardiac valve, and/or with the aid of a balloon. The release device 160 or the inner shaft is then withdrawn into the outer shaft and the insertion device 120 is removed from the body. The implant 155 remains fully positioned in the body (not shown).

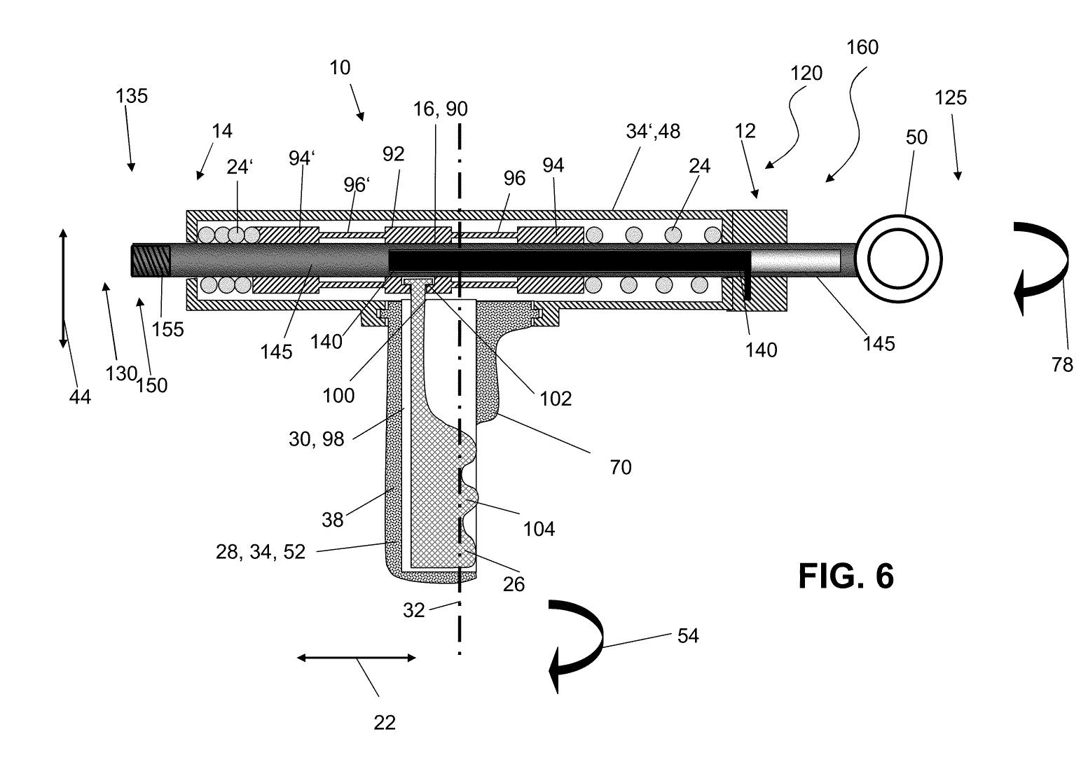

The operating mode of the covering of the implant 155 with the outer insertion element 145, what is known as a retrieval mode, will now be described on the basis of FIGS. 5A, 5B and 6. This mode can be used for example for assembly of the implant 155 in the insertion device 120 or for withdrawal or renewed covering of the implant 155 during the implantation process in the event of incorrect positioning or incorrect function of the implant 155. The forces necessary for this are much greater than with the release, since the implant 155, which is partly unfolded for example, has to be brought again to its compressed or folded small diameter. The retrieval mode is set by turning the control element 28 or the first grip segment 34 (pistol grip 52) in the peripheral direction 54 until the lettering 58 “RETRIEVE” appears in the display 60 of the display element 38 (see FIG. 5B). The pommel 70, against which a palm (not shown) now rests, and the operating surface 104 thus point in the direction of the proximal end 12 in order to symbolize that the outer insertion element 145 will be moved in the direction of the distal end 14 (see FIGS. 5A and 5B). In this setting, the sleeve 92 is in its central position in the body 10 and the spring elements 24, 24′ are substantially hardly compressed.

In this position of the first grip segment 34, the eccentric active element 30 or the recess 98 is arranged such that a clearance for sinking the actuator 26 is provided in the direction of the distal end 14. If the force-locked connection between the force transfer region 90 and the outer insertion element 145 is closed by means of the clamping means and the actuator 26 or the control lever is then axially displaced in the direction of the distal end 14, it enters completely into the recess 98 in the first grip segment 34 (see FIG. 6). The sleeve 92 is thus also moved in the direction of the distal end 14 and entrains the outer insertion element 145 via the captive connection in the direction of the distal end 14. This occurs until the spring element 24′ is fully compressed, which limits the movement of the actuator 26. At the catheter tip 150, the outer insertion element 145 is thus advanced toward the catheter tip 150. If the implant 155 is axially blocked by the inner insertion element 140, the outer insertion element 145 thus slides (again) over the implant 155 and covers said implant.

If, once the force-locked connection has been released, the actuator 26 or the control lever is then disengaged, the spring element 24′, which also represents a return spring, pushes the sleeve 92 with the force transfer region 90 and the actuator 26 back in the direction of the proximal end 12 as far as its central position or its starting position. This process can be repeated until the outer insertion element 145 completely covers the implant 155 again. Whether the implant 155 can be drawn back into the release device 160 can be determined by means of the marking on the outer insertion element 145. As a result, the implant 155 is placed in the outer insertion element 145 and the assembly process is concluded.

A third operating mode for a fast release of the implant 155, what is known as a fast release mode, is shown in FIGS. 7A and 7B. The fast release mode is activated by releasing the captive connection between the sleeve 92 and the outer insertion element 145. This is only possible with a releasable force-locked connection and/or interlocking connection between the sleeve 92 and the outer insertion element 145. With an embodiment of the release device 160 with an integrally bonded connection between the sleeve 92 and the outer insertion element 145, a fast release mode is not possible. The force-locked connection and/or interlocking connection for example could be released, alternatively to the above-described variant with the clamping means, by forming the outer insertion element 145 within the body 10 with different widths or diameters over its contour or its periphery in the peripheral direction 78. In the deploy or retrieval mode, the interlocking connection and/or force-locked connection between the sleeve 92 and the outer insertion element 145 exists over a larger width or a larger diameter of the outer insertion element 145. If the body 10 or the release device 160 and the insertion elements 140, 145 are then rotated against one another in the peripheral direction 78 of the body 10 by approximately 90°, a smaller width or a smaller diameter of the outer insertion element 145 assumes the position of the larger width or of the larger diameter (not shown in detail). The interlocking connection and/or force-locked connection between the sleeve 92 and the outer insertion element 145 thus releases so that the outer insertion element 145 can be moved freely relative to the force transfer region 90. The end regions 94, 94′ of the transfer element 16 are formed as sliding regions 94, 94′ in order to convey a sliding movement of the transfer element 16 to the outer insertion element 145.

In order to display the operating mode then set, the lettering 58 “FAST RELEASE” is displayed in the window 68 of the display 60 (see FIG. 7B). This can occur as a result of an aperture that is moved as a result of the rotation of the body 10 in the peripheral direction 78, whereby the lettering 58 appears in the window 68. The operator can then pull the operating element 50 at the proximal end of the outer insertion device 145 for fast release of the implant 155 (see arrow in FIG. 7A). The outer insertion element 145 thus moves in the direction of the proximal end 12 and the implant 155 is released (not shown).

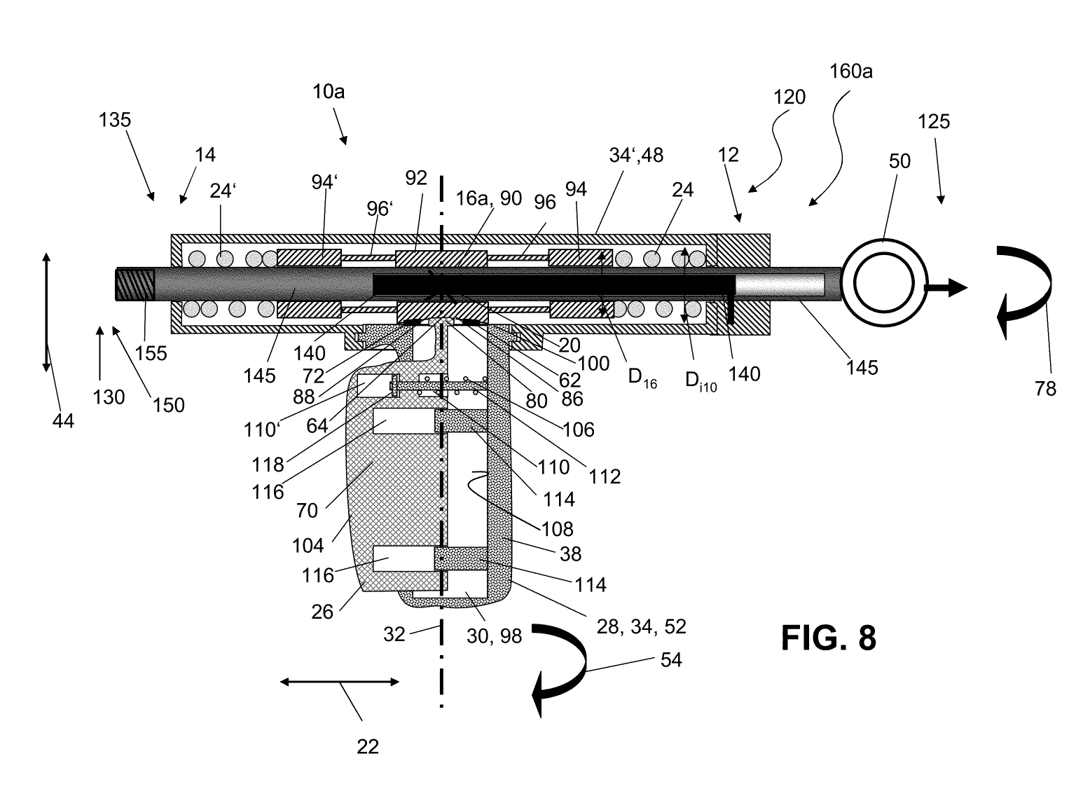

Two alternative exemplary embodiments of the body 10 and of the release device 160 are illustrated in FIGS. 8 to 12. Like components, features and functions are basically denoted in principle by like reference signs. To distinguish between the exemplary embodiment in FIGS. 8 to 12 and that in FIGS. 1 to 7 however, the letters a or b have been added to the reference signs of differently designed components in the exemplary embodiment of FIGS. 8 to 12. The following description is basically restricted to the differences from the exemplary embodiment in FIGS. 1 to 7, wherein reference can be made to the description of the exemplary embodiment in FIGS. 1 to 7 with regard to like components, features and functions.

The release device 160a or the body 10a in FIGS. 8 to 11 differs from the body 10 and the release device 160 in FIGS. 1 to 7 in that a transfer element 16a can convey a releasable force-locked connection between the transfer element 16a and the outer insertion element 145 by means of a specific embodiment of the force transfer region 90. The transfer element 16a is formed as a sleeve 92 arranged coaxially with the insertion elements 140, 145 of an insertion device 120 and extending around the body 10a in the peripheral direction 78. To convey the force-locked connection, the force transfer region 90 is equipped with two chamfers 86, 88, which run outwardly at an incline from a central region 64 in the radial direction 44. An actuator 26 axially displaceable in the longitudinal direction 22 in the body 10a has a contact face 80 at its radially upper end, said contact face bearing against the region 64 in an unactuated state of the actuator 26 (see FIG. 8). A slit 100, which acts as a rail, is provided in housing 48 of the body 10a in order to support the actuator 26 and to guide said actuator axially (see also FIG. 2).

A starting position of the release device 160a for a slow release of an implant 155, that is to say the deploy mode, is shown in FIG. 8. Here, all components are arranged substantially similarly to the illustration in FIG. 3A. If the actuator 26 is then actuated or displaced in the direction of a proximal end 12 of the body 10a, the contact face 80 of the actuator 26 moves along the chamfer 86 in the direction of the proximal end 12, whereby the axis 20 of the force transfer region 90 is tilted in an anticlockwise direction.

It is possible to tilt the force transfer region 90 relative to end regions 94, 94′ of the sleeve 92 since a transition region 96, 96′ is arranged axially between the force transfer region 90 and each of the end regions 94, 94′ and has a thinner material thickness than the force transfer region 90 and the end regions 94, 94′. The transition regions 96, 96′ thus have a lower flexural rigidity than the force transfer region 90 and the end regions 94, 94′, whereby it is possible to tilt the force transfer region 90 without transferring said tilt to the end regions 94, 94′. When tilting the force transfer region 90, the transition regions 96, 96′ of the sleeve 92 are bent in a clockwise direction. The transfer element 16a has a diameter D₁₆ that is selected so that the transfer element 16 is arranged with play for a tilting movement of the transfer element 16 relative to an inner diameter Duo of the body 10a (see FIG. 8).

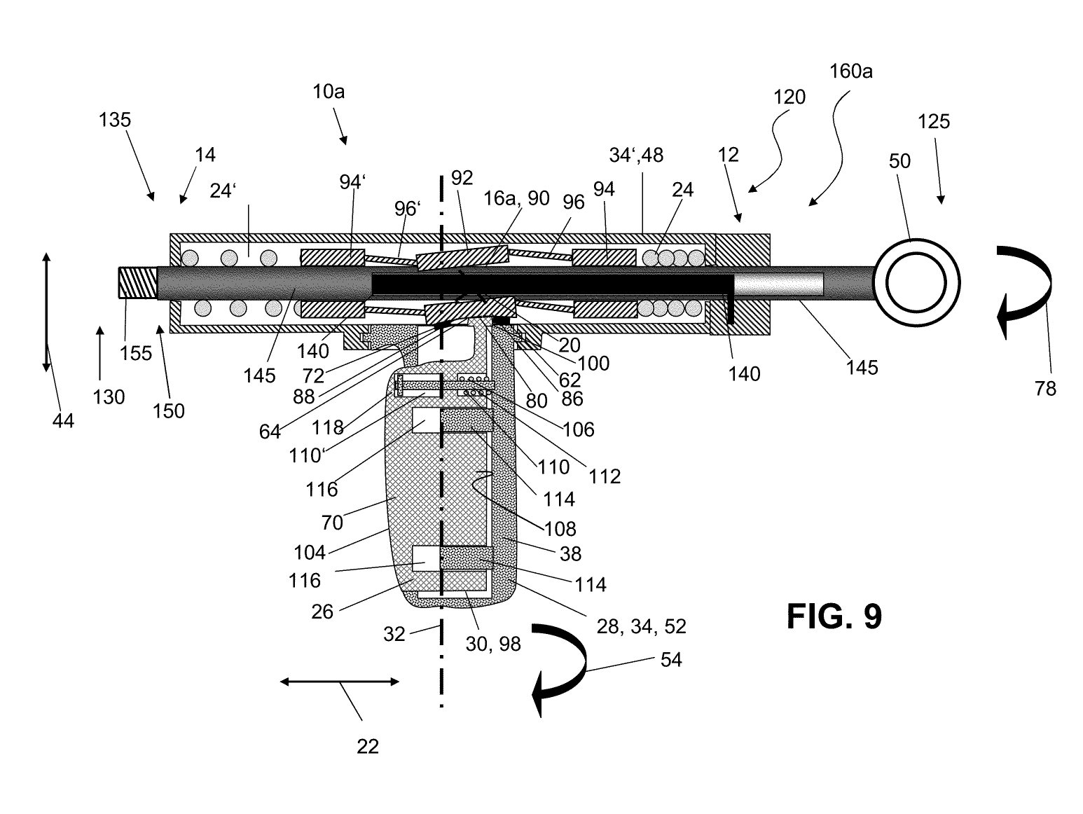

In this tilted position, the force transfer region 90 establishes a force-locked connection with the outer insertion element 145, whereby the transfer element 16a can be tilted in an anticlockwise direction for the release of the implant 155. So as to then move the outer insertion element 145 in the direction of the proximal end 12, the transfer element 16a has a stop 62 for the actuator 26. If the actuator 26 then impacts the stop 62, it pushes the force transfer region 90 in the direction of the proximal end 12 as it moves further in the direction of the proximal end 12. Due to the force-locked connection between the force transfer region 90 and the outer insertion element 145, said insertion element is also slid in the direction of the proximal end 12. In this case, a spring element 24 arranged axially between the end region 94 of the sleeve 92 and the proximal end 12 is compressed (see FIG. 9).

When the actuator 26 is displaced in the direction of the proximal end 12 of the body 10a, the actuator 26 is biased by a spring element 106, for example in the form of a spiral spring. This spring element 106 extends in its substantially relaxed state between a vertical inner wall 108 of the grip segment 34, which points in the direction of the receptacle 98, and a vertical wall of a proximal recess 110 in the actuator 26 (see FIG. 8). A distal end of the spring element 106 thus impacts the wall of the proximal recess 110. To support the spring element 106, it is placed around a bearing element 112, which is arranged or integrally molded on the inner wall 108 of the grip segment 34. The bearing element 112 extends substantially parallel to the direction of movement of the actuator 26 and spans the corresponding proximal recess 110.

To axially guide the actuator 26 homogeneously, the inner wall 108 has two guide webs 114, which are vertically spaced and oriented parallel to one another and to the direction of movement of the actuator 26. Each guide web 114 engages in a corresponding recess 116 in the actuator 26. As can be seen in FIG. 9, the movement of the actuator 26 in the direction of the proximal end 12 of the body 10a is limited by an impact of a vertical end web 118 of the bearing element 112 against a distal wall of a distal recess 110′ in the actuator 26. In order to ensure a harmonic, matched and intended movement of the actuator 26, the orientations and dimensions, for example length, width, etc., of the bearing element 112, the guide webs 114 and the recesses 110, 110′, 112 are adapted to one another.

In principle, the spring element 106 could also be fixed axially between the radially upper end of the actuator 26 and the stop 62. In this case, a second spring element 106 would have to be provided for the covering of the implant 155 (see below) and would have to be axially fixed between the radially upper end of the actuator 26 and a stop 72 (not shown).

If the actuator 26 is then disengaged, it moves back into its starting position (see FIG. 8) as a result of a resilience of the spring element 106, guided by the slit 100. The force-locked connection between the force transfer region 90 and the outer insertion element 145 is thus released, whereby said force transfer region 90 can again assume its position coaxial with the insertion elements 140, 145. The spring element 24, which is also a return spring, can expand and presses the sleeve 92 with the force transfer region 90 in the longitudinal direction 22 and in the direction of the distal end 14 back into the central position or the starting position.

The actuator 26 thus conveys both the intended movement in the longitudinal direction 22 of the transfer element 16a for the intended relative movement in the longitudinal direction 22 between the first and the second insertion element 140, 145 and a tilting movement of the transfer element 16a, or the outer shaft is withdrawn relative to the inner shaft and therefore also to the implant 155 as a result of the actuation of the actuator 26. The implant 155 is thus exposed and expanded (see FIG. 9). The release device 160a or the inner shaft is then withdrawn into the outer shaft and the insertion device 120 is removed from the body. The implant 155 remains fully positioned in the body (not shown).

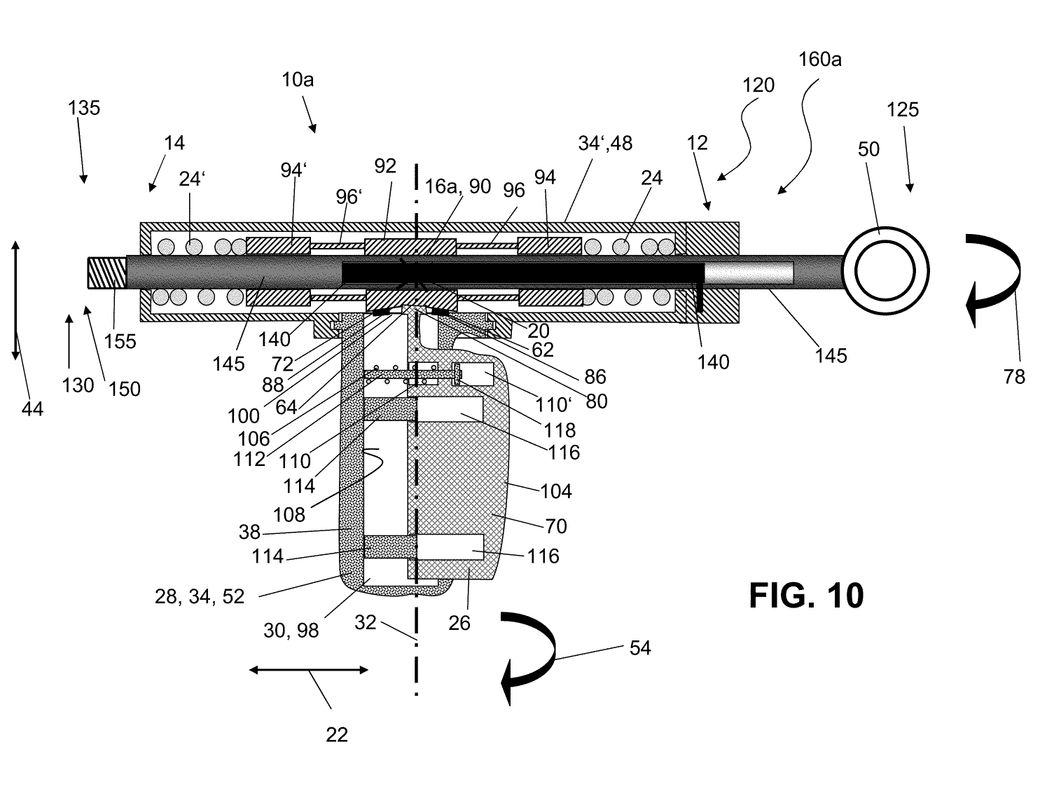

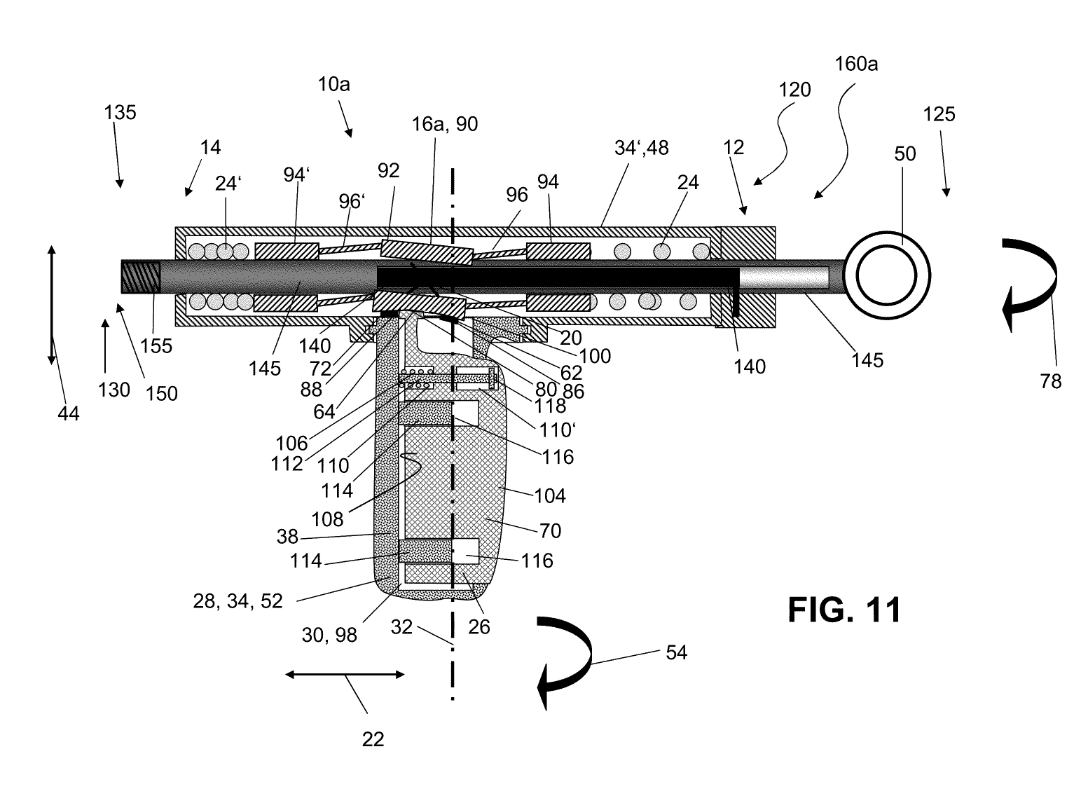

A starting position of the release device 160a for covering the implant 155, that is to say the retrieval mode, is shown in FIG. 10. In this case, all components are arranged substantially similarly to the illustration in FIG. 5A. This mode is set by rotating the first grip segment 34 about its axis of rotation 32. If the actuator 26 is then actuated or displaced in the direction of a distal end 14 of the body 10a, the contact face 80 of the actuator 26 is moves along the chamfer 88 in the direction of the distal end 14, whereby the axis 20 of the force transfer region 90 is tilted in a clockwise direction. In this tilted position, the force transfer region 90 establishes a force-locked connection with the outer insertion element 145, whereby the transfer element 16a is tiltable in a clockwise direction for the covering of the implant 155. So as to then move the outer insertion element 145 in the direction of the distal end 14, the transfer element 16a has a stop 72. If the actuator 26 then impacts against the stop 72 and the actuator 26 is moved further in the direction of the distal end 14, the actuator 26 slides the force transfer region 90 in the direction of the distal end 14. Due to the force-locked connection between the force transfer region 90 and the outer insertion element 145, said insertion element is also slid in the direction of the distal end 14. In this case, a spring element 24′, which is arranged axially between the end region 94′ of the sleeve 92 and the distal end 14, is compressed (see FIG. 11). At a catheter tip 150, the outer insertion element 145 is thus advanced toward the catheter tip 150. If the implant 155 is axially blocked by the inner insertion element 140, the outer insertion element 145 thus slides (again) over the implant 155 and covers said implant.

If the actuator 26 is then disengaged, it moves back into its starting position, guided by the slit 100, as a result of the resilience of the spring element 106. The force-locked connection between the force transfer region 90 and the outer insertion element 145 is thus released and the force transfer region 90 can again assume its position coaxial with the insertion elements 140, 145. The spring element 24′, which is also a return spring, can expand and presses the sleeve 92 with the force transfer region 90 in the longitudinal direction 22 and in the direction of the distal end 14 back into the central position or the starting position.

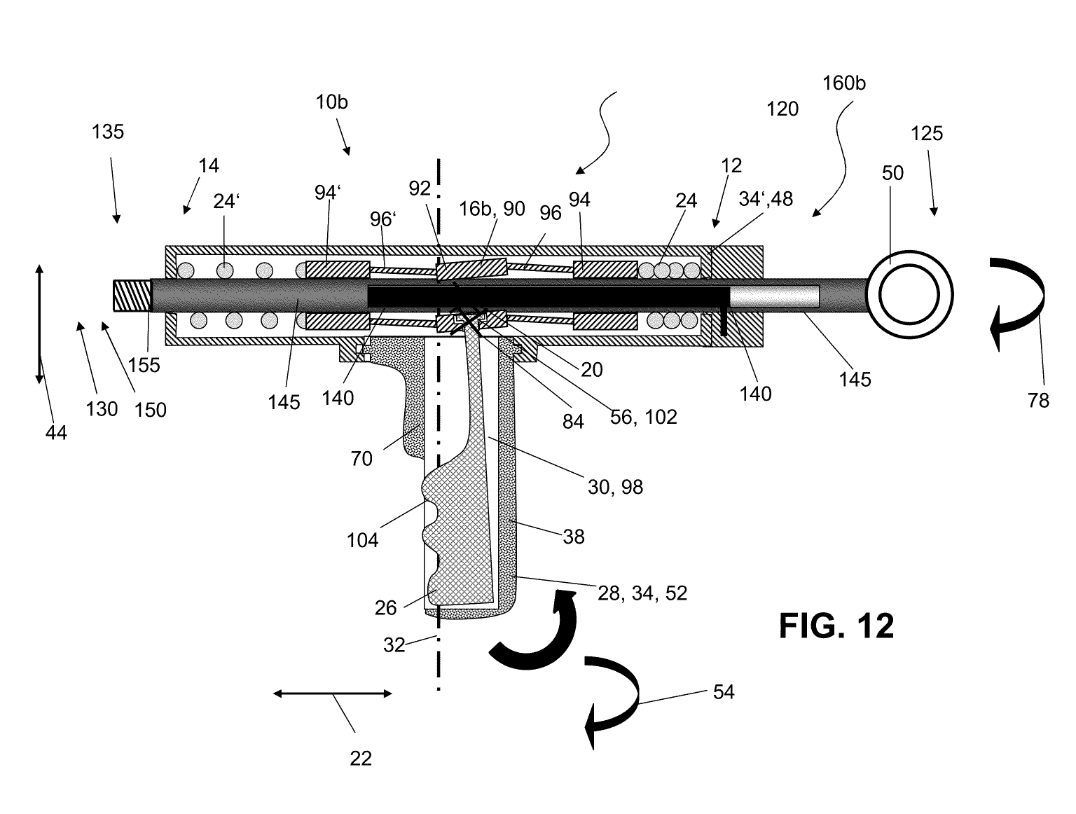

The release device 160b or the body 10b in FIG. 12 differs from the body 10 and the release device 100 in FIGS. 1 to 7 in that a releasable force-locked connection between the transfer element 16b and the outer insertion element 145 is conveyed by tilting an actuator 26 together with a transfer element 16b and on the basis of a force transfer region 90.

For this purpose, the axially displaceable actuator 26 in the form of a control lever in the first grip segment 34 is pivotably mounted via a contact point 56 in a receptacle 102 in the force transfer region 90. The contact point 56 is a pivot axis 84 of the actuator 26, which is aligned parallel to an axis of rotation 20 of the transfer element 16b and is arranged at the same axial height as the axis of rotation 32 of the first grip segment 34 in a starting position of the unactuated actuator 26, in which a sleeve 92 with the force transfer region 90 is arranged in a central position in a body 16b (not shown in detail, arrangement similar to the arrangement in FIG. 1).

In a starting position, which is set before implantation of an implant 155, the axis 20 of the force transfer region 90 is fixed axially between two spring elements 24, 24′ and extends coaxially with the insertion elements 140, 145 of an insertion device 120 (not shown in detail, arrangement similar to the arrangement in FIG. 1). If the actuator 26 is then pivoted in an anticlockwise direction in the contact point 56 (see the arrow in FIG. 12), the transfer element 16b is also tilted in an anticlockwise direction about its axis 20. This is made possible due to the reduced flexural rigidity of transition regions 96, 96′ of the sleeve 92 and therefore due to the deflection thereof in a clockwise direction. The actuator 26 thus produces a force-locked connection to the outer insertion element 145, whereby said insertion element is axially fixed relative to the body 10b and the inner insertion element 140. Once maximum tilt has been reached, the actuator 26 displaces the tilted transfer element 16b or the sleeve 92 together with the outer insertion element 145 axially in the direction of the proximal end 12. This occurs until the spring element 24 is completely compressed, which limits the axial movement of the actuator 26 (see FIG. 12).

By disengaging the actuator 26, said actuator moves back into its unactuated position or the starting position, whereby the transfer element 16b also returns to its position coaxial with the insertion elements 140, 145 (not shown). As a result, the force-locked connection between the force transfer region 90 and the outer insertion element 145 is removed. The spring element 24, which is formed as a return spring, thus expands again and the sleeve 92 is slid in the longitudinal direction 22 and in the direction of the distal end 14 into the central position or the starting position (not shown).

The actuation of the actuator 26 causes the outer shaft to withdraw relative to the inner shaft and therefore also to the implant 155. The implant 155 is thus exposed and expanded (see FIG. 12). The release device 160b or the inner shaft is then withdrawn into the outer shaft and the insertion device 120 is removed from the body. The implant 155 remains fully positioned in the body (not shown).

A retrieval mode can be implemented once the first grip segment 34 has been rotated about the axis of rotation 32. This mode is triggered by pivoting the actuator 26 and therefore the transfer element 16b in a clockwise direction and progresses with the specifications of the exemplary embodiment described in FIG. 12 substantially similarly to the progression of the exemplary embodiment described in FIGS. 10 and 11, whereby reference is hereby made to this description.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### REFERENCE LIST

10 body
12 end
14 end
16 transfer element
20 axis
22 longitudinal direction
24 spring element
26 actuator
28 control element
30 active element
32 axis of rotation
34 grip segment
36 receptacle
38 display element
40 passage
42 passage
44 radial direction
46 anchoring
48 housing
50 operating element
52 pistol grip
54 peripheral direction
56 contact point
58 lettering
60 display
62 stop
64 region
66 disc
68 window
70 pommel
72 stop
78 peripheral direction
80 contact face
84 pivot axis
86 chamfer
88 chamfer
90 force transfer region
92 sleeve
94 region
96 transition region
98 recess
100 slit
102 receptacle
104 operating surface
106 spring element
108 inner wall
110 recess
112 bearing element
114 guide web
116 recess
118 end web
120 insertion device
125 end
130 end
135 shaft region
140 insertion element
145 insertion element
150 catheter tip
155 implant
160 release device
D diameter
Di inner diameter
M material thickness

What is claimed is:

1. A release device for releasing a medical implant from an insertion device, with which the implant can be released by a relative movement between a first and a second insertion element, the release device comprising a body with a proximal end, which is remote from a distal end of the insertion device during use, and with a distal end, which faces the distal end of the insertion device during use, wherein a first grip segment, a second grip segment and at least one actuator are provided between the proximal and the distal end, wherein the at least one actuator is manually operable and can be brought substantially into at least the first grip segment substantially axially displaceably in order to effect an intended relative movement in the longitudinal direction between the first and the second insertion element of the insertion device.

2. The release device as claimed in claim 1, wherein an axis of rotation of the first grip segment runs substantially perpendicularly to at least one of the insertion elements and/or the at least second grip segment runs substantially coaxially with at least one of the insertion elements.

3. The release device as claimed in claim 1, wherein the intended relative movement in the longitudinal direction between the first and the second insertion element of the insertion device is achieved at least by means of a force-locked connection, in particular a releasable force-locked connection, between the at least one actuator and one of the insertion elements.

4. The release device as claimed in claim 1, having a control element, with which an operating mode can be set and/or can be displayed, wherein the control element has an active element, preferably for changing between at least two operating modes and in particular arranged eccentrically to an axis of rotation of the control element.

5. The release device as claimed in claim 4, wherein the first grip segment is formed in one piece with the control element.

6. The release device as claimed in claim 1, having a display element for displaying a set operating mode, wherein the display element is preferably formed in one piece with a rotatable grip segment.

7. The release device as claimed in claim 1, having at least one substantially axially displaceable transfer element, which transfers the axial movement of the at least one actuator upon actuation of the at least one actuator to at least one of the insertion elements in order to effect the intended relative movement in the longitudinal direction between the first and the second insertion element of the insertion device.

8. The release device as claimed in claim 7, wherein the at least one transfer element has a cylindrical sleeve, which is optionally arranged substantially coaxially with at least one of the insertion elements.

9. The release device at least as claimed in claim 7, wherein the at least one transfer element has at least one force transfer region and at least one sliding region, wherein the at least one sliding region preferably conveys a sliding movement of the at least one transfer element to one of the insertion elements.

10. The release device as claimed in claim 9, wherein the at least one transfer element comprises at least one transition region, which is preferably arranged axially between the at least one force transfer region and the at least one sliding region, and/or wherein the at least one transition region and the at least one force transfer region each have a radial material thickness, wherein in particular the material thickness of the at least one transition region is thinner than the material thickness of the at least one force transfer region.

11. The release device at least as claimed in claim 7, wherein the at least one transfer element is biased by at least one spring element, wherein the at least one transfer element can be returned into its starting position by the at least one spring element.

12. The release device at least as claimed in claim 7, wherein, for a fast release of the implant, the at least one transfer element is substantially coaxially adjustable relative to at least one of the insertion elements.

13. The release device as claimed in claim 1, wherein an axial movement of the at least one actuator in the direction of the proximal end of the body conveys a release of the implant and/or an axial movement of the at least one actuator in the direction of the distal end of the body conveys a covering of the implant by one of the insertion elements.

14. The release device as claimed in claim 1, wherein the body, has at least one receptacle for an inner insertion element in order to immovably fix the inner insertion element to the body.

15. The release device as claimed in claim 1, wherein an insertion element protruding from the body has at least one marking, which is intended to indicate the extent to which the implant has been released and/or whether the implant can be withdrawn into the release device.

16. An insertion device for inserting a medical implant, which can be released by a relative movement between a first and a second insertion element, said insertion device comprising a release device for releasing the medical implant, in particular as claimed in of claim 1, said release device comprising a body with a proximal end, which is remote from a distal end of the insertion device during use, and with a distal end, which faces the distal end of the insertion device during use, wherein a first grip segment, a second grip segment and at least one actuator are provided between the proximal and the distal end, wherein the at least one actuator is manually operable and can be brought substantially into at least the first grip segment substantially axially displaceably in order to effect an intended relative movement in the longitudinal direction between the first and the second insertion element of the insertion device.
